(19) United States
(12) Patent Application Publication
(10) Pub. No.: US 2025/0049306 A1
(43) Pub. Date: Feb. 13, 2025

(54) ELEVATORS FOR MEDICAL DEVICES AND RELATED METHODS

(71) Applicant: Boston Scientific Scimed, Inc., Maple Grove, MN (US)

(72) Inventors: Evan WILDER, Malden, MA (US); Sean POWELL, Holden, MA (US)

(73) Assignee: Boston Scientific Scimed, Inc., Maple Grove, MN (US)

(21) Appl. No.: 18/799,498

(22) Filed: Aug. 9, 2024

Related U.S. Application Data
(60) Provisional application No. 63/518,661, filed on Aug. 10, 2023.

(51) Int. Cl. A61B 1/005; A61B 1/00; A61B 1/01; A61B 1/018

(52) U.S. Cl. CPC A61B 1/0055; A61B 1/00098; A61B 1/01; A61B 1/018

(57) ABSTRACT

A medical device including a handle and a distal tip. The distal tip includes an elevator configured to change an orientation of an accessory device. The elevator is rotatable about a first axis and a second axis. Rotation of the elevator about the first axis raises or lowers the elevator. Rotation of the elevator about the second axis angles the elevator to the right or left. The medical device includes a first wire attached to a first side of the elevator and a second wire attached to a second side of the elevator. Moving the first wire and second wire the same distance proximally or distally relative to the distal tip rotates the elevator about the first axis. Moving one of the first wire and second wire a distance relative to the other of the first wire and second wire rotates the elevator about the second axis.

### CROSS-REFERENCE TO RELATED APPLICATIONS

This application claims the benefit of priority to U.S. Provisional Application No. 63/518,661, filed on Aug. 10, 2023, which is incorporated by reference herein in its entirety.

### TECHNICAL FIELD

Various aspects of this disclosure relate generally to medical devices having steerable components. In particular, aspects of this disclosure pertain to steerable elevators and distal tip features of duodenoscopes or other types of medical devices, among other aspects.

### BACKGROUND

A duodenoscope or other type of medical device may include a handle and a sheath, and the sheath may be insertable into a body lumen of a subject. The sheath may terminate in a distal tip portion, which may include features such as optical elements (e.g., camera, lighting), air/water outlets, and working channel openings. An elevator may be disposed at the distal tip portion and may be actuatable in order to change an orientation of an accessory device passed through the working channel. For example, the elevator may be pivotable or otherwise movable.

Elements in the handle of the duodenoscope may control the elements of the distal tip of the duodenoscope. For example, buttons, knobs, levers, etc. may control elements of the distal tip. The elevator may be controlled via a control mechanism, such as a lever, of the handle. The control mechanism may be attached to a control wire that attaches to the elevator. When the control mechanism (e.g., the lever) is actuated, the wire may move proximally and/or distally, thereby raising and/or lowering the elevator, respectively. The elevator may move with one degree of freedom, moving an accessory device extending from the working channel up and down. However, such an elevator does not allow for steering the accessory device in other directions. Therefore, a need exists for a medical devices and related methods that utilize elevators that facilitate the movement of the accessory device.

### SUMMARY

Each of the aspects disclosed herein may include one or more of the features described in connection with any of the other disclosed aspects. Aspects of the disclosure may relate to steerable medical device elevators that change an orientation of an accessory device along multiple axes.

An exemplary medical device may include a handle and a distal tip. The distal tip may include an elevator configured to change an orientation of an accessory device. The elevator may be rotatable about a first axis and a second axis. The first axis differs from the second axis.

In some aspects, rotation of the elevator about the first axis may raise and lower the elevator. Rotation of the elevator about the second axis may move the elevator about a longitudinal axis of a body of the elevator. In some aspects, the medical device further includes a first wire or cable coupled to a first side of the elevator and a second wire or cable coupled to a second side of the elevator. Movement of the first cable relative to the second cable may rotate the elevator about the second axis.

In some aspects, the medical device may comprise a roll bar. A third wire may be coupled to the roll bar. A fourth wire may be coupled to the roll bar. Movement of the third wire may be configured to rotate the elevator about the first axis. Movement of the fourth wire may be configured to rotate the elevator about the second axis. The first wire may be coupled to the roll bar at a first connection point. The second wire may be coupled to the roll bar at a second connection point. The third wire may be coupled to the roll bar at a third connection point. The third connection point may be equidistant from the first connection point and the second connection point.

In some aspects, the handle of the medical device may include a first actuator coupled to the first cable and a second actuator coupled to the second cable. The first actuator and second actuator may be actuatable independently of one another.

In some aspects, proximal or distal movement of a single wire may be configured to rotate the elevator of the medical device about both the first axis and the second axis. The distal tip of the medical device may include a housing defining a cavity, wherein the elevator includes an axle received within the cavity, and wherein the cavity has an approximately cylindrical shape.

In some aspects, the elevator of the medical device may include an axle defining a channel, wherein the distal tip further includes a rotatable shaft having a projection, and wherein the channel is configured to receive the projection. The first axis may be a longitudinal axis of a shaft of the rotatable shaft. The second axis may be a longitudinal axis of the projection or the channel.

In some aspects, the elevator of the medical device may include an approximately biconical axle.

In some aspects, the elevator of the medical device may include an axle. The distal tip may include a housing defining a cavity, wherein the axle is received within the cavity, wherein the cavity is configured to allow a central portion of the axle to rotate about a longitudinal axis of the axle and to prevent the central portion of the axle from translating within the cavity. The cavity may be configured to allow ends of the axle to translate within the cavity.

Another exemplary medical device may comprise a distal tip. The distal tip may include an elevator configured to change an orientation of an accessory device. The medical device may include a handle. The handle may include a first actuator coupled to a first control wire or cable and a second actuator coupled to a second control wire or cable. Each of the first actuator and the second actuator may be configured to rotate the elevator about at least one axis.

In some aspects, the first control wire or cable may be coupled to a first side of the elevator and the second control wire or cable may be coupled to a second side of the elevator.

In some aspects, each of the first actuator and the second actuator may be separately actuatable.

Another exemplary medical device may include a distal tip. The distal tip may comprise an elevator having an axle and a body. The distal tip may further comprise at least one wire or cable coupled to the elevator. The at least one wire or cable may be configured to rotate the elevator about a longitudinal axis of the axle and about a longitudinal axis of the body.

In some aspects, the at least one wire or cable may be at least two wires or cables.

### BRIEF DESCRIPTION OF THE FIGURES

The accompanying drawings are incorporated in and constitute a part of this specification. These drawings illustrate aspects of the disclosure that, together with the written descriptions herein, serve to explain this disclosure. Each drawing depicts one or more exemplary aspects according to this disclosure, as follows:

FIG. 1A depicts an exemplary medical device.

FIG. 1B depicts a proximal end of a handle of the medical device of FIG. 1A.

FIG. 2 depicts a distal end of an exemplary medical device, with an elevator of the distal end in a first configuration.

FIG. 3 depicts the distal end of FIG. 2 with the elevator in a second configuration.

FIG. 4 depicts a portion of the distal end of FIG. 2 with the elevator in a third configuration.

FIG. 5 depicts a portion of the distal end of FIG. 2 with the elevator in a fourth configuration.

FIG. 6 depicts a portion of the distal end of FIG. 2 with the elevator in a fifth configuration.

FIG. 7 depicts a portion of the distal end of FIG. 2 with the elevator in a sixth configuration.

FIG. 8 depicts a portion of the elevator and a portion of a housing of the distal end of FIG. 2.

FIG. 9 depicts a portion of an alternative elevator and a portion of an alternative housing.

FIG. 10 depicts a portion of an alternative elevator and a portion of an alternative housing.

FIG. 11A and FIG. 11B depict a distal end of an exemplary medical device.

FIG. 12A and FIG. 12B depict portions of an alternative elevator shown in FIGS. 11A and 11B.

FIG. 13 depicts a distal end of another exemplary medical device

FIG. 14 depicts an elevator of an exemplary medical device.

FIG. 15 depicts a rotatable shaft for use with the elevator of FIG. 14.

FIG. 16 depicts a portion of a distal end of a medical device with elevator of FIG. 14 in a first configuration.

FIGS. 17A and 17B depict aspects of the distal end of FIG. 16 with the elevator in a second configuration.

FIGS. 18A and 18B depict aspects of the distal end of FIG. 16 with the elevator in a third configuration.

FIGS. 19A and 19B depict aspects of the distal end of FIG. 16 with the elevator in a fourth configuration.

FIGS. 20A and 20B depict aspects of the distal end of FIG. 16 with the elevator in a fifth configuration.

FIGS. 21A and 21B depict aspects of the distal end of FIG. 16 with the elevator in a sixth configuration.

FIG. 22 diagrams an exemplary movement of the elevator FIG. 14 moving between the first through sixth configurations depicted in FIGS. 16 through 21B.

### DETAILED DESCRIPTION OF THE FIGURES

Reference will now be made in detail to examples of the present disclosure described above and illustrated in the accompanying drawings. Wherever possible, the same reference numbers will be used throughout the drawings to refer to the same or like parts.

The terms “proximal” and “distal” are used herein to refer to the relative positions of the components of an exemplary medical device. When used herein, “proximal” refers to a position relatively closer to the exterior of the body or closer to a medical professional using the medical device. In contrast, “distal” refers to a position relatively further away from the medical professional using the medical device, or closer to the interior of the body. As used herein, the terms “comprises,” “comprising,” “has,” “having,” “includes,” “including,” or other variations thereof, are intended to cover a non-exclusive inclusion, such that a device or method that comprises a list of elements does not include only those elements, but may include other elements not expressly listed or inherent thereto. Unless stated otherwise, the term “exemplary” is used in the sense of “example” rather than “ideal.” As used herein, the terms “about,” “substantially,” and “approximately,” indicate a range of values within +/−10% of a stated value.

A duodenoscope or other medical device may be utilized to perform a medical procedure at a treatment site within a body lumen of a subject. In procedures such as endoscopic retrograde cholangiopancreatography (ERCP), an operator may navigate the medical device to a duodenum of a subject, and utilize an elevator of a distal tip of the medical device to change an orientation of one or more accessory devices (e.g., tomes, guidewires, or the like) in order to, e.g., cannulate a papilla of the subject. Disclosed herein are distal tips of medical devices that include an elevator that is movable in multiple directions. In some examples, multiple control wires may be coupled to the elevator, and the control wires may be separately movable (as well as movable in unison). In other examples, a single control wire may be utilized to move the elevator in a plurality of directions. Although duodenoscopes and ERCP may be referenced in particular herein, it will be appreciated that other types of medical devices (e.g., medical devices used for endoscopic ultrasound (EUS)) may also include the disclosed aspects.

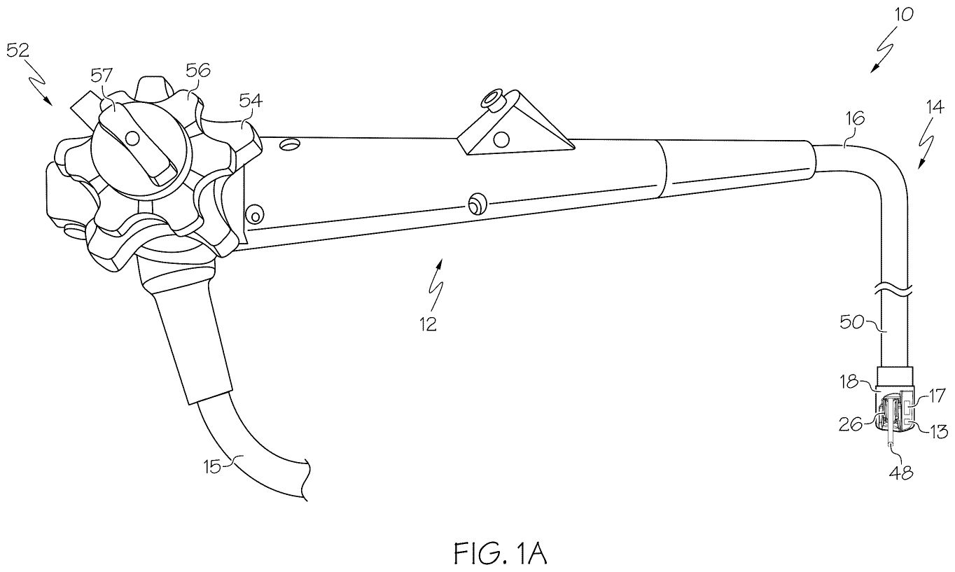

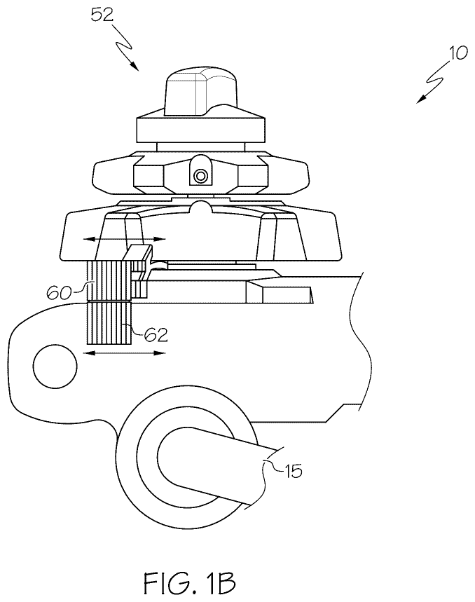

FIG. 1A depicts an exemplary medical device 10 having a handle 12 and an insertion portion 14. FIG. 1B shows a proximal end of handle 12. Medical device 10 may also include an umbilicus 15 for purposes of connecting medical device 10 to sources of, for example, air, water, suction, power, etc., as well as to image processing and/or viewing equipment. Although duodenoscopes are particularly referenced herein, the disclosure also encompasses other types of devices, such as endoscopes, bronchoscopes, gastroscopes, EUS scopes, colonoscopes, ureteroscopes, bronchoscopes, laparoscopes, cystoscopes, aspiration scopes, sheaths, catheters, or similar devices. A reference to a duodenoscope herein should be understood to encompass any of the above medical devices.

Insertion portion 14 may include a sheath or shaft 16 and a distal tip 18. Distal tip 18 may include an imaging device 13 and a lighting source 17. Distal tip 18 may further include an elevator 26 at a distal end of a working channel of the medical device 10, for changing an orientation of an accessory device or tool 48 inserted in the working channel of medical device 10. Elevator 26 may alternatively be referred to as a swing stand, pivot stand, raising base, or any suitable other term.

A distal portion of shaft 16 that is connected to distal tip 18 may have a steerable section 50. Steerable section 50 may include, for example, an articulation joint. Shaft 16 and steerable section 50 may include a variety of structures which are known or may become known in the art.

Handle 12 may have one or more actuators/control mechanisms 52. One or more of the control mechanisms 52 may provide control over steerable section 50 or may allow for provision of air, water, suction, etc. For example, handle 12 may include control knobs 54, 56 for left, right, up, and/or down control of steerable section 50. For example, one of knobs 54, 56 may provide left/right control of steerable section 50 and the other of knobs 54, 56 may provide up/down control of steerable section 50. Handle 12 may further include one or more locking mechanisms 57 (e.g. knobs or levers) for preventing steering and/or braking of steerable section 50 in at least one of an up, down, left, or right direction.

As seen in FIG. 1B, handle 12 may include one or more elevator control levers 60, 62. Although two elevator control levers 60, 62 are depicted, it will be appreciated that alternative numbers of levers (e.g., one lever or three or more levers) may be utilized. Elevator control levers 60, 62 may rotate or pivot independently of one another and/or may rotate or pivot in tandem with one another. Each lever 60, 62 may control (e.g., be coupled to) a cable or wire connected to the respective lever 60, 62. The cable or wire connected to each lever 60, 62 may be directly or indirectly connected to a portion of elevator 26 or another suitable structure (e.g., as described below for FIGS. 11A-11B). Actuating levers 60 and/or 62 may move one or more cables proximally or distally and thereby actuate elevator 26, as described in further detail below. An operator may use a finger to span both levers 60, 62 to move levers 60, 62 in tandem. In embodiments with two or more cables actuated by levers 60, 62, depressing or pulling levers 60, 62 together may, relative to handle 12, distally extend or proximally retract the two or more cables equally. The levers 60, 62 may be selectively locked together to more easily depress and pull the levers 60, 62 the same distance (i.e., move levers 60, 62 in tandem), thereby extending and retracting the two or more cables equally.

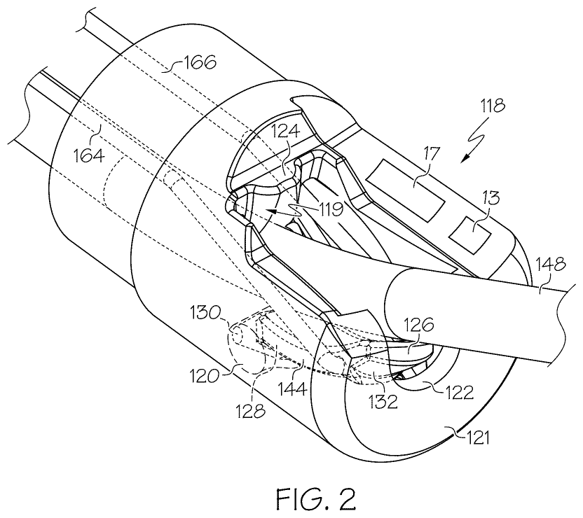

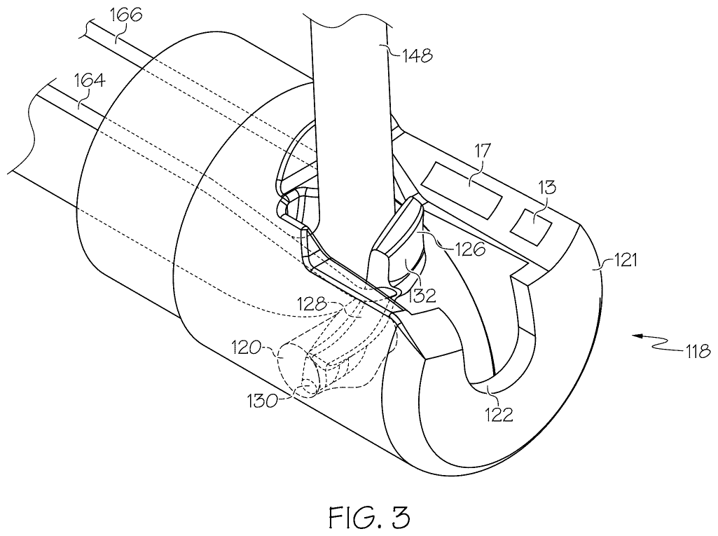

FIGS. 2-8 depict a distal tip 118 in various configurations. Distal tip 118 may be used instead of distal tip 18 with medical device 10 and may have any of the features of distal tip 18. Certain elements of distal tip 118 (e.g. caps, covers, sheaths, etc.) may be omitted from FIGS. 2-8 to more clearly illustrate aspects of the disclosure. Distal tip 118 may include an elevator 126. Elevator 126 may be configured to receive and contact an accessory device 148 that extends distally from a distal opening 119 (see FIG. 3) of a working channel extending through a shaft (not shown in FIGS. 2-8 but having any of the features of shaft 16) of medical device 10. Elevator 126 may have any of the features of elevator 26, discussed above.

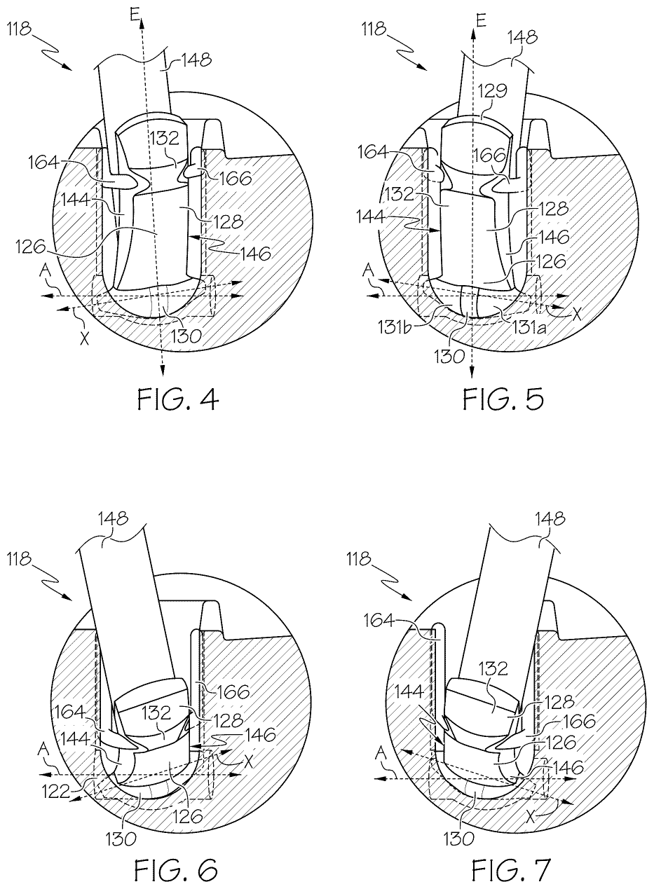

Elevator 126 may include a body 128 and an axle 130. When elevator 126 is lowered/open (see, e.g., FIG. 2) body 128 may extend distally from axle 130 generally along a longitudinal axis of elevator 126. Body 128 may include a guide surface that faces distal opening 119 and is sized and shaped to receive a portion of accessory device 148. Body 128 may have any feature of a body of any elevator known in the art. In a lowered/open configuration of elevator 126 (a configuration in which elevator 126 has not been actuated, as shown in FIG. 2), an axis X of axle 130 (labeled in FIGS. 4-7) may extend along a generally lateral direction (a direction that is approximately perpendicular to a longitudinal axis of medical device 10 and/or body 128).

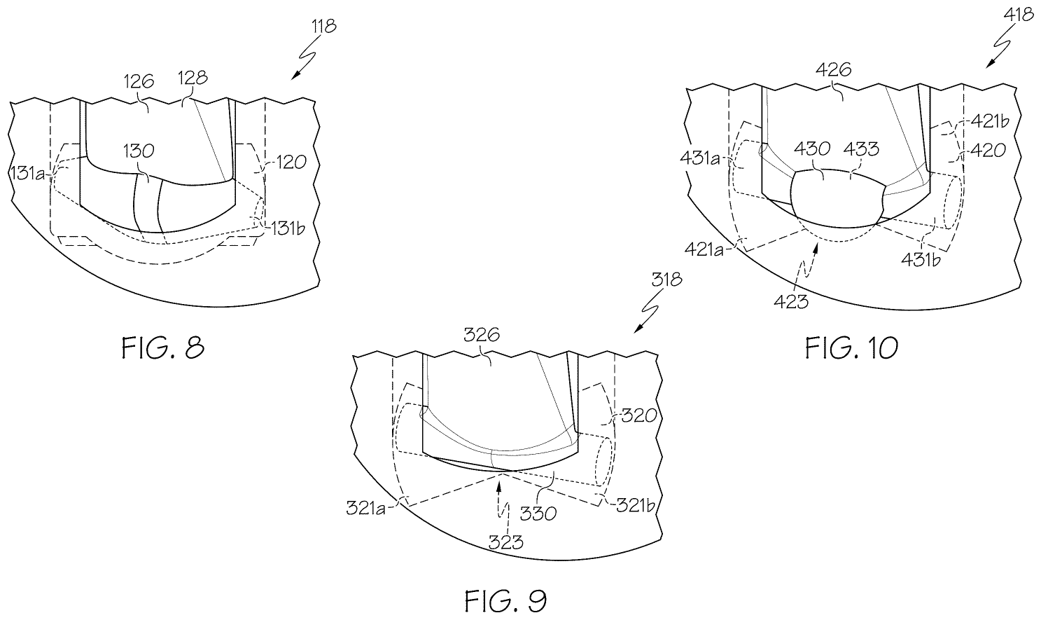

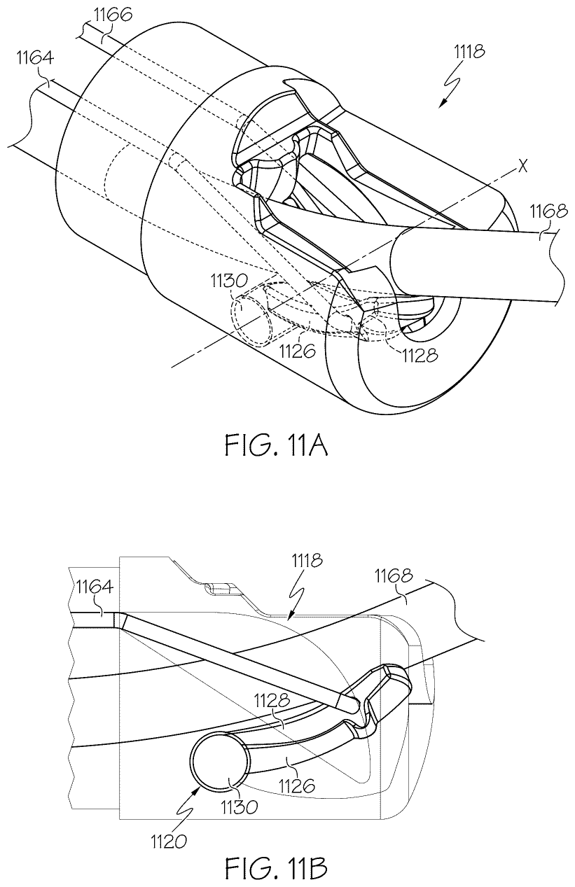

Axle 130 may be biconical in shape, as shown in FIGS. 2-8. As best shown in FIG. 8, axle 130 may include a first portion 131a and a second portion 131b. First portion 131a and second portion 131b (see FIG. 5) may each be shaped like a cone or a frustum. As shown in the FIGS. 2-8, first portion 131a and second portion 131b may be frustum-shaped (and may lack points). However, first portion 131a and second portion 131b may have pointed ends such that they are cone-shaped. It will be appreciated that shapes described herein are not limited to exact shapes and include approximations of the shapes. Each of first portion 131a and second portion 131b may narrow as it extends outward along axis X from a central longitudinal axis of elevator 126 (i.e., from a center of axle 130). Laterally outer portions of axle 130 may be narrower than a central portion of axle 130. In other words, starting from one side of axle 130 and moving along axis X of axle 130, axle 130 may widen (e.g., along portion 131a) and then narrow again (e.g., along portion 131b). Bases of frustums/cones of first portion 131a and second portion 131b may be coupled to one another near or at a center of axle 130. In some examples, bases of frustums/cones of first portion 131a and second portion 131b may be coupled directly to one another. In other examples, a segment having a cylindrical or other shape may extend between the bases of frustums/cones of first portion 131a and second portion 131b. As will be discussed below, axle 130 is not limited to a biconical shape and may be any number of shapes; other exemplary shapes will be discussed in greater detail with respect to FIGS. 9 and 10. Axle 130 may have a greater lateral width (In a direction perpendicular to the longitudinal axis of elevator 126) than body 128, as shown, for example, in FIGS. 4 and 5.

Distal tip 118 (e.g., a housing 121 of distal tip 118) may include a cavity 120. Cavity may extend generally perpendicularly to a longitudinal axis of device 10/distal tip 118. In some examples, cavity 120 may be approximately cylindrical in shape and may extend along an axis A (see FIGS. 4-7). Axle 130 may be positioned within cavity 120. Axle 130 may translate and rotate within cavity 120. A lateral width of cavity 120 (in a direction approximately perpendicular to a longitudinal axis of device 10/distal tip 118 and along axis A) may be slightly greater than a lateral width of axle 130 along axis X in order to accommodate axle 130. A diameter of cavity 120 may be slightly larger than a diameter of axle 130 at a center of axle 130 (e.g., at a widest portion of axle 130). Housing 121 may be formed from a transparent material.

A first wire or cable 164 and a second wire or cable 166 may be coupled to elevator 126. A proximal end (not shown) of first cable may be coupled (directly or indirectly) to one of levers 60, 62, and a proximal end (not shown) of second cable 166 may be coupled (directly or indirectly) to the other of levers 60, 62. A distal end of first cable 164 may be connected to a portion of elevator 126, such as a distal surface 132 of body 128, as shown particularly in FIGS. 4-7, near a first lateral side 144 of elevator 126. A distal end of second cable 166 may be connected to distal surface 132 of body 128 near a second lateral side 146 of elevator 126, opposite first lateral side 144. In some examples, first cable 164 and second cable 166 may formed integrally (i.e., from a single piece of material) or may be coupled to one another at their distal ends, so as to form a loop. In such examples, the loop may be fixedly coupled to distal surface 132. Moving first cable 164 and/or second cable 166 using levers 60 and/or 62 may move elevator 126 or at least a portion of elevator 126, as discussed in further detail below.

FIGS. 2-7 show elevator 126 in various configurations. FIGS. 2, 3 depict perspective views of distal tip 118 with elevator 126 in a lowered (open) configuration and a raised (closed) configuration, respectively and show how a distal tip 129 (labeled in FIG. 5) of elevator 126 moves up and down (hereinafter referred to as a “pitch” of elevator 126). FIGS. 4 and 5 illustrate perspective cross-sections of distal tip 118 showing elevator 126 in a raised-rotated configuration, angling a raised accessory device 148 to the left and right, respectively, when viewing distal tip 118 in a proximal direction. FIGS. 6 and 7 illustrate perspective cross-sections of distal tip 118 in a lowered-rotated configuration, angling a lowered accessory device to the left and right, respectively, when viewing distal tip 118 in the proximal direction. FIGS. 4-7 illustrate, in addition to pitch of the elevator, the rotational range of motion (hereinafter referred to as the “roll”) of elevator 126 about a longitudinal axis of the body 128. It should be understood that elevator 126 is not limited to these configurations shown in FIGS. 2-7 and that the configurations shown in FIGS. 2-7 are merely shown to illustrate a range of motion of elevator 126. Elevator 126 may, for example, have configurations that are combinations of the configurations of FIGS. 2-7.

FIG. 2 depicts elevator 126 of the distal tip 118 in a first configuration. The first configuration is a lowered (open) configuration (i.e. at the lowest pitch). In the lowered configuration of the elevator 126, the distal tip 118 may be configured to receive an accessory device 148 that is extended distally from opening 119. In the first configuration, accessory device 148 may extend generally distally. While in the lowered configuration, a longitudinal axis of elevator 126 may be approximately parallel to a longitudinal axis of distal tip 118 or may have an open, oblique angle with respect to a longitudinal axis of distal tip 118. Distal tip 118 may include a depression 122, which may be configured to receive accessory device 148 when elevator 126 is in the lowered configuration of FIG. 2. In the lowered configuration of FIG. 2, levers 60, 62 may be in line with one another, and distal ends of cables 164, 166 may extend a same distal amount.

FIG. 3 depicts elevator 126 of the distal tip 118 in a second configuration. The second configuration is a raised (closed) configuration (i.e. at the highest pitch). To move elevator 126 from the first configuration (FIG. 2) to the second configuration (FIG. 3), and operator may move levers 60, 62 in tandem in a first direction to move cables 164, 166 proximally and thereby exert a proximal force on elevator 126, rotating it about axis X of axle 130 (not labeled in FIGS. 2-3 but labeled in FIGS. 4-7). Axis X of axle 130 may be approximately parallel to axis A of cavity 120 in the first and second configurations. An operator may move elevator 126 from the second configuration to the first configuration by moving levers 60, 62 in a second direction, opposite the first direction, in order to move cables 164, 166 distally, thereby exerting a distal force on elevator 126. The arrows in FIG. 1B depict the directions in which levers 60, 62 may be moved.

Raising elevator 126 from the first configuration to the second configuration may change an orientation of accessory device 148. In the second configuration (FIG. 3), accessory device 148 may extend generally radially outward. While in the raised configuration, a longitudinal axis of elevator 126 may be approximately perpendicular to a longitudinal axis of distal tip 118. Distal tip 118 may include a boss 124 extending distally. In the raised configuration shown in FIG. 3 or the rotated-raised configurations shown in FIGS. 4 and 5, the accessory device 148 may contact boss 124, and accessory device 148 may be locked between boss 124 and elevator 126. Boss 124 and a guide surface of elevator 126 may have generally complementary surfaces to facilitate locking.

FIGS. 4 and 5 depict elevator 126 of FIG. 2 in a third configuration and a fourth configuration, respectively. The third configuration is a left-rotated raised configuration and the fourth configuration is a right-rotated raised configuration. For example, a transition from the second configuration (FIG. 3) to the third configuration (FIG. 4) may be achieved by moving first cable 164 (e.g., via lever 60) slightly distally and second cable 166 (e.g., via lever 62) slightly proximally and a transition from the second configuration to the fourth configuration (FIG. 5) may be achieved by moving second cable 166 slightly distally and first cable 164 slightly proximally. A transition from the first configuration (FIG. 2) to the third configuration (FIG. 4) may be achieved by moving both first cable 164 and second cable 166 proximally, with second cable 166 moving further proximally than first cable 164. To transition from the first configuration to the third configuration, levers 60, 62 may be moved independently or may be moved in tandem for a first part of the stroke and independently for a second part of the stroke. A transition from the first configuration (FIG. 2) to the fourth configuration (FIG. 5) may be achieved by moving both first cable 164 and second cable 166 proximally, with first cable 164 moving further proximally than second cable 166.

As shown in FIGS. 4 and 5, in the third and fourth configurations, axis X of axle 130 may be offset from axis A of cavity 120. In other words, axis X of axle 130 may be transverse (at an angle to) axis A of cavity 120. In FIG. 4, axis X may extend slightly upward in the coordinate system of FIG. 4 on second lateral side 146. Axis X also may extend slightly into the page of FIG. 4 on second lateral side 146. In FIG. 5, axis X may extend slightly upward in the coordinate system of FIG. 5 on first lateral side 144. Axis X also may extend slightly into the page of FIG. 5 on first lateral side 144.

Proximally moving one of the cables 164, 166 relative to the other cable 164, 166 rotates elevator 126, and axle 130, about axis E, the longitudinal axis of the body 128 (as shown in FIGS. 4 and 5). The geometry of cavity 120 may limit a maximum angle of rotation of the axle 130, thereby limiting the roll of the elevator 126 (i.e. a maximum angle that the elevator may be rotated left or right about axis E). Limiting the roll of elevator 126 limits, by extension, a maximum angle that accessory device 148 may be rotated left or right relative to the longitudinal axis of distal tip 118. In some embodiments, the roll of the elevator may be limited to angles that are in the field of vision of the imaging device 13. Limiting the roll of the elevator to angles that are in the field of vision of the imaging device 13 increases the overall safety of the device as the accessory device and it associated medical instruments and tools would always be observable by an operator.

FIGS. 6, 7 depict elevator 126 of FIG. 2 in a fifth and sixth configuration, respectively. The fifth configuration is a left rotated lowered configuration and the sixth configuration is a right rotated lowered configuration. For example, a transition from the first configuration to the fifth configuration may be achieved by moving second cable 166 proximally relative to first cable 164. A transition from the first configuration to the sixth configuration may be achieved by moving first cable 164 proximally relative to second cable 166. A transition from the third configuration (FIG. 4) to the sixth configuration (FIG. 7) may be achieved by moving the first cable 164 and second cable 166 distally and moving first cable 164 proximally relative to second cable 166 until axle 130 has rotated from left to right about the axis E. The transitions provided above are merely exemplary and are provided for the sake of illustration. Other transitions among the other configurations are within the scope of this disclosure.

As shown in FIGS. 6 and 7, in the fifth and sixth configurations, axis X of axle 130 may be offset from axis A of cavity 120. In other words, axis X of axle 130 may be transverse (at an angle to) axis A of cavity 120. In FIG. 6, axis X may extend slightly upward in the coordinate system of FIG. 6 on second lateral side 146. Axis X also may extend slightly into the page of FIG. 6 on second lateral side 146. In FIG. 7, axis X may extend slightly upward in the coordinate system of FIG. 7 on first lateral side 144. Axis X also may extend slightly into the page of FIG. 7 on first lateral side 144.

Surfaces of cavity 120 and their dimensions may partially restrict movement and rotation of axle 130 within cavity 120. In other words, cavity 120 may define boundaries for movement of axle 130. Rotation of axle 130 about axis A of cavity 120 (when axis X is approximately coaxial with axis A) may be unrestricted by cavity 120, allowing for elevator 126 to transition from a lowered configuration (as shown in FIG. 2) to a raised configuration (as shown in FIG. 3). Such rotation may occur when an operator moves levers 60, 62 in tandem to move cables 164, 166 a same amount proximally or distally. When levers 60, 62 are moved separately or are moved different amounts from one another, rotation of axle 130 about axis E may be partially restricted by the surfaces of cavity 120 limiting a roll of elevator 126. In some embodiments, the rotation of the roll of elevator 126 may be partially restricted by contacting the geometry of distal tip 118.

In alternative aspects, lever 60, 62 may be a single lever or other type of actuator. Below, lever 60 is referenced, although it will be appreciated that lever 62 may alternatively be a single lever. A shaft or other structure may connect lever 60 to cables 164, 166 (e.g., to proximal ends of cables 164, 166). Lever 60 may be able to be depressed and raised with respect to handle 12 (e.g., may translate into and out of the page of FIG. 1B). Depressing or raising lever 60 may adjust lengths of (extend distally or retract proximally) both cables 164, 166 equally or approximately equally. For example, the shaft coupled to cables 164, 166 may move proximally or distally, thereby moving both of cables 164, 166 proximally or distally. Rotation of the lever 60 or a rotatable element of shaft 60 may rotate the shaft coupled to cables 164, 166, thereby adjusting lengths of the cables 164, 166 relative to each other (i.e., move one of cables 164, 166 further distally of the other of cables 164, 166) and thus achieve non-planar motion of the elevator, as shown in FIGS. 4-7.

In alternative aspects, a first of cables 164, 166 may be fixedly coupled to elevator 126 and may be extended proximally or distally to rotate elevator 126 about axle 130 to open/close elevator 126 (i.e., raise or lower elevator 126 to adjust a pitch of elevator 126). The second of cables 164, 166 may be coupled to elevator 126 near a location where the first of cables 164, 166 is coupled. Elevator 126 and/or a housing 121 of distal tip 118 may have angled surfaces that may be used to rock elevator 126 left or right (adjusting a roll of elevator 126) as tension is applied to the second of cables 164, 166. This may allow an operator to control independently both a pitch of elevator 126 and a roll of elevator 126. For example, one of levers 60, 62 (coupled to the first of cables 164, 166) may control pitch, and the other of levers 60, 62 (coupled to the second of cables 164, 166) may control roll.

FIGS. 8-10 depict exemplary axles of elevators disposed within a cavity of a housing of a distal tip. FIG. 8 depicts a portion of elevator 126 of distal tip 118. Axle 130 of elevator 126 may be positioned within cavity 120 of distal tip 118.

FIG. 9 depicts a portion of an elevator 326 of a distal tip 318. An axle 330 of elevator 326 may be positioned within a cavity 320 of distal tip 318. Elevator 326 may have any of the features of elevators 126, 26. Axle 330 may be approximately cylindrical in shape. Cavity 320 may be an hourglass-like shape, with a narrower central portion 323 and wider side portions 321a, 321b. Cavity 320 may have a shape similar to two frustums joined together at their narrower ends. Central portion 323 may have a diameter that is slightly larger than a diameter of axle 330, such that sides of axle 330 may be movable within cavity 320, while a central portion of axle 330 may be rotatable about an axis of axle 330 but not translatable within cavity 320. Sides of axle 330 may be able to move within side portions 321a, 321b, such that elevator 326 may achieve the configurations described for elevator 126, above (see FIGS. 2-7).

FIG. 10 depicts a portion of an elevator 426 of a distal tip 418. Elevator 426 may have any of the features of elevators 326, 126, 26. An axle 430 of the elevator 426 may be positioned within a cavity 420 of distal tip 418. Axle 430 may be formed so as to have cylindrical side portions 431a, 431b and a rounded central portion 433. Central portion 433 may have a shape like a portion of sphere. Central portion 433 may be positioned at a midpoint of axle 430. Central portion 433 may protrude outward relative to side portions 431a, 431b. Cavity 420 may have a shape similar to a bowtie, with frustum-shaped side portions 421a, 421b and a rounded central portion 423. Side portions 421a, 421b may have similar shapes to side portions 321a, 321b. Central portion 423 may have a shape that is complementary to central portion 433 of axle 130. Central portion 433 may be rotatable about an axis of axle 430 but not translatable within cavity 320. Side portions 431a, 431b may be movable within side portions 421a, 421b to achieve configurations described for elevator 126 in FIGS. 2-7. As compared with distal tip 418, central portion 423 may provide increased retention of central portion 433 in a desired position within cavity 420. It should be understood that some geometries of the axles and bodies, such as those described, may be less expensive to manufacture, may be stronger, may require less material or labor, and/or may have lower torque generated by friction for improved user performance. For example, axles and bodies that have a smaller contact diameter may generate lower frictional torque.

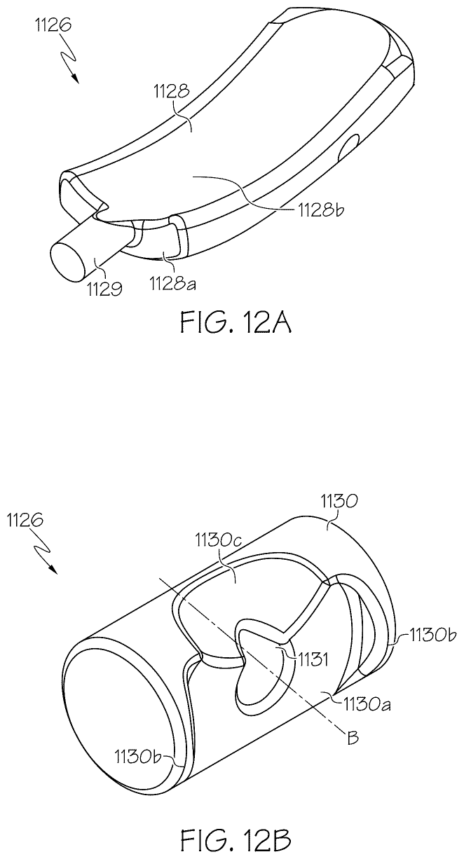

FIGS. 11A and 11B depicts a distal tip 1118 of another exemplary medical device. Distal tip 1118 may have any of the features distal tips 18, 118, 318, 418, 518 or any of the other distal tips described below. Distal tip 1118 is depicted with an accessory device 1168 passed therethrough. Distal tip 1118 may include an elevator 1126. Elevator 1126 may have any of the features of elevators 126, 326, 426, 526, or any of the elevators described below. Elevator 1126 may include a body 1128 and an axle 1130 (shown in detail in FIGS. 12A and 12B, respectively). Distal tip 1118 may include a cavity 1120. Cavity 1120 may extend generally perpendicularly to a longitudinal axis of distal tip 1118. In some examples, cavity 1120 may be approximately cylindrical in shape and may extend along an axis X. Axle 1130 may be positioned within cavity 1120. Axle 1130 may rotate within cavity 1120 about axis X but may not translate within cavity 1120. In some examples, axle 1130 may be approximately cylindrical in overall shape. A lateral width of cavity 1120 (in a direction approximately perpendicular to a longitudinal axis of distal tip 1118 and along axis X) may be slightly greater than a lateral width of axle 1130 along axis X in order to accommodate axle 1130.

As shown in FIG. 12A, body 1128 may include a protrusion 1129 extending from a proximal surface 1128a of body 1128. Axle 1130 may include a hole 1131 sized and shaped to receive protrusion 1129. Protrusion 1129, and by extension: body 1128, may rotate about a longitudinal axis of hole 1131 or a longitudinal axis of the body 1128, both hereinafter referred to as axis B (labeled in FIG. 12B). Hole 1131 may be defined through a surface 1130a (e.g., a planar surface) of axle 1130. Surface 1130a may be flush with proximal surface 1128a when protrusion 1129 is received within hole 1131. Planar surface 1130a may be complementary to proximal surface 1128a. The surfaces of axle 1130 may be configured so that body 1128 may rotate about axis B unimpeded. For example, planar surface 1130a may include extensions 1130b that accommodate proximal surface 1128a as body 1128 rotates about axis B. Body 1128 may include a guide surface 1128b configured to receive at least a portion of accessory device 1168. Axle 1130 may include a guide surface 1130c configured to receive a portion of accessory device 1168 that serves as continuation of guide surface 1128b of body 1128. Guide surface 1130c of axle 1130 may be adjacent to planar surface 1130a.

With reference to FIGS. 11A and 11B, elevator 1126 may be coupled to a first cable 1164 and a second cable 1166 using any of the mechanisms described above for elevator 126 and cables 164, 166. First cable 1164 and second cable 1166 may have any of the properties of cables 164 and 166 and may be actuated using any of the mechanisms described above.

As mentioned above, surfaces of cavity 1120 and their dimensions may restrict translation of axle 1130. In other words, cavity 1120 may define boundaries for movement of axle 1130. Rotation of axle 1130 about axis X of cavity 1120 may be unrestricted by cavity 1120, allowing for elevator 1126 to transition from a lowered configuration (similar to the lowered configuration shown in in FIG. 2) to a raised configuration (similar to the raised configuration shown in FIG. 3). Such rotation may occur when an operator moves levers 60, 62 in tandem to move cables 1164, 1166 a same amount proximally or distally.

When levers 60, 62 are moved separately or are moved different amounts from one another, rotation of body 1128 about axis B may occur. Rotation about axis B allows for accessory device 1168 to rotate left and right, similarly to as shown in FIGS. 4-7, however, unlike elevator 126 shown in FIGS. 4-7, axle 1130 of elevator 1126 would not rotate along with body 1128 about axis B. The rotation of body 1128 may be partially restricted by the surfaces/geometry of distal tip 1118 and/or surface 1130a of axle 1130, limiting a roll of elevator 1126.

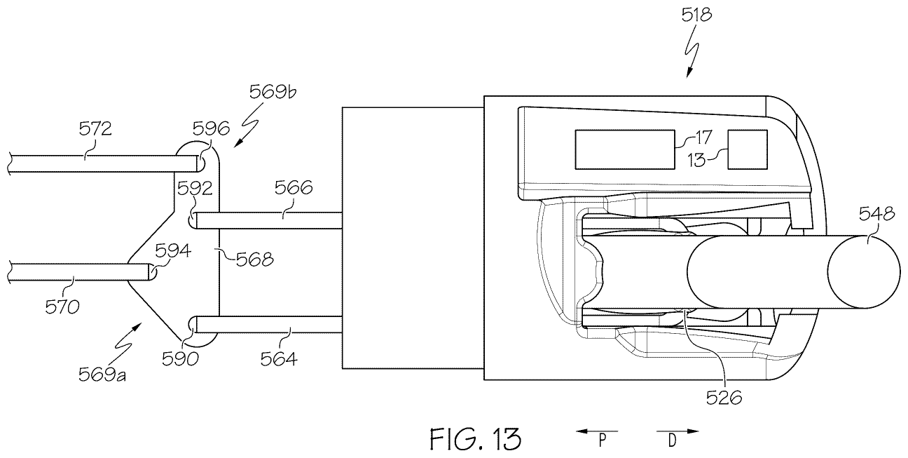

FIG. 13 depicts a distal tip 518 of another exemplary medical device. Various features of distal tip 518 (e.g. caps, covers, sheaths, etc.) may be omitted from FIG. 13 to more clearly illustrate aspects of the disclosure. Distal tip 518 may be used in conjunction with medical device 10, described above. Distal tip 518 and elevator 526 may include any of the features of distal tips 418, 318, 118, 18 or elevators 426, 326, 126, 26, respectively. Elevator 526 may be configured to receive and contact an accessory device 548. Elevator 526 may be coupled to a first cable 564 and a second cable 566 using any of the mechanisms described above for elevator 126 and cables 164, 166. First cable 564 may include any of the features of first cable 164, and second cable 566 may include any of the features of second cable 166.

First cable 564 and second cable 566 may be coupled to a roll bar 568. For example, as shown in FIG. 13, first cable 564 and second cable 566 extend proximally from elevator 526 to roll bar 568 and are connected to roll bar 568 at different as 590, 592 respectively. In an unactuated (e.g., open) state of elevator 526, connection points 590, 592 may be at a same longitudinal distance (i.e., a distance in the proximal/distal direction). Although roll bar 568 is shown at a distal portion of a medical device (e.g., at or near distal tip 518), it will be appreciated that roll bar 568 may be disposed within handle 12 or proximal portions of shaft 16.

Roll bar 568 may be coupled to a pitch cable 570 and a roll cable 572. For example, roll bar 568 may be coupled to distal ends of pitch cable 570 and roll cable 572. Pitch cable 570 may be connected to a first lever of the handle (e.g., one of levers 60, 62), and roll cable 572 may be connected to a second lever of the handle (e.g., the other of levers 60, 62). Pitch cable 570 may be connected to roll bar 568 at connection point 594. Connection point 594 may be equidistant from connection points 590 and 592. For example, connection point 594 may be proximal of a midpoint of a line segment drawn between connection points 590 and 592. Connection points 590, 592, and 594 may be vertices of an isosceles triangle (e.g., an equilateral triangle). Roll cable 572 may be connected to roll bar 568 at connection point 596. Connection point 596 may be positioned such that a straight line extends through connection points 590, 592, and 596. Connection point 596 may be positioned to the left of, or near, connection point 590 or to the right of, or near, connection point 592. Connection point 596 may be to one side of connection points 590, 592—that is, connection point 596 may not be in between connection points 590, 592. As shown, a distance between connection points 590, 592 may be greater than a distance between connection point 596 and connection point 592 (which is closer to connection point 596 than connection point 590 is). However, such an arrangement is merely exemplary. A benefit to roll bar 568 is that the system will naturally self-center if triangle geometry is used as shown in the exemplar and described above. Furthermore, the rate of roll can be more accurately engineered as it is not dependent on simultaneously adjusting pitch as described in other examples within this disclosure.

As shown in FIG. 13, roll bar 568 may have a first portion 569a that has a shape that is approximately a triangle with rounded vertices. In alternatives, first portion 569a may have one or more pointed vertices or may have any other suitable shape. Connection points 590, 592, 594 may be disposed on first portion 569a. Connection point 594 may be disposed near a proximal vertex of first portion 569a, with connection points 592, 594 being at distal vertices of first portion 569a. Roll bar 568 may also have a second portion 569b that protrudes from one side of first portion 569a. As shown in FIG. 13, a distal edge of roll bar 568 may be straight, extending across first and second portions 569a, 569b. First portion 569a may have a greater length in a proximal/distal direction, such that a proximal portion of first portion 569a may extend further in a proximal direction than a proximal portion of second portion 569b. The shape of roll bar 568 described above is merely exemplary, however, and any suitable shape may be utilized.

Pitch cable 570 may be proximally moved (shortened within shaft 16) to adjust the pitch of elevator 526, transitioning elevator 526 from a lowered configuration to a raised configuration. When pitch cable 570 is moved proximally, roll bar 568 may be moved proximally, thereby moving first cable 564 and second cable 566 proximally by an equal distance. Pitch cable 570 may also be moved distally (lengthened within shaft 16) to adjust the pitch of elevator 526, transitioning elevator 526 from a raised configuration to a lowered configuration. When pitch cable 570 is moved distally, roll bar 568 may be moved distally, thereby moving cables 564, 566 distally by an equal amount.

Roll cable 572 may be proximally retracted or distally extended to change the position of one of first cable 564 or second cable 566 relative to the other cable 564, 566. For example, retracting roll cable 572 proximally may cause second cable 566 to move proximally and first cable 564 to move distally, thereby rotating elevator 526 to the left when viewed in the proximal direction (similarly as shown in FIGS. 4 and 6). Similarly, distal movement of roll cable 572 may move first cable 564 proximally and second cable 566 distally, thereby rotating elevator 526 to the right when viewed in the proximal direction (similarly as shown in FIGS. 5 and 7).

FIG. 13 also includes a distal direction arrow labeled ‘D’ and a proximal direction arrow labeled ‘P’ relative to distal tip 518. These proximal and distal direction arrows are applicable to any distal tip described within this disclosure (e.g. distal tips 618, 518, 418, 318, 118).

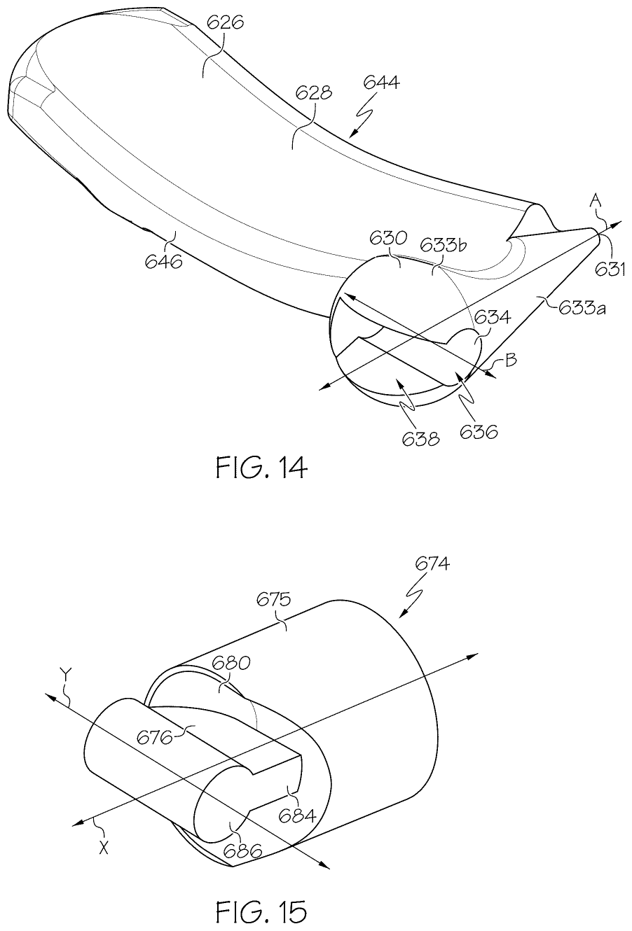

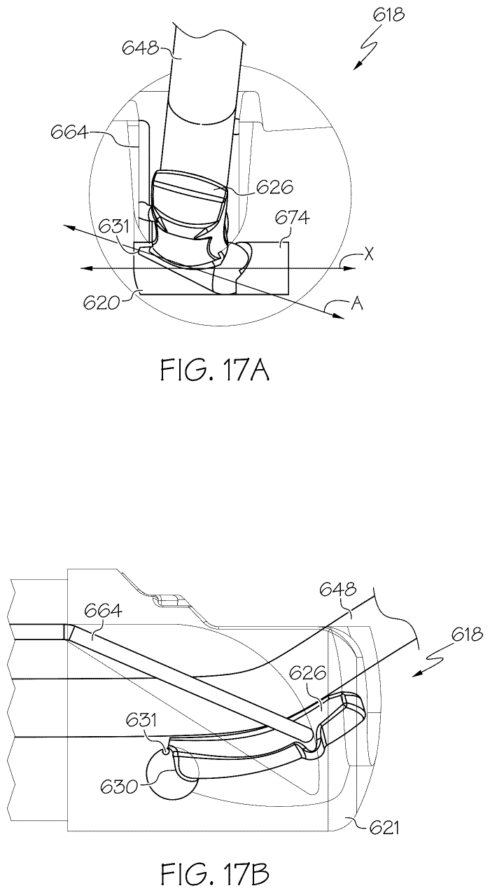

FIGS. 14-21B depict aspects of an alternative distal tip 618 (see, e.g., FIG. 17B), which may be used in conjunction with device 10, instead of distal tip 18. Distal tip 618 may include any feature of distal tips 518, 418, 318, 118, 18. Distal tip 618 may include an elevator 626. As shown in FIG. 14, elevator 626 may include a body 628 and an axle 630. Body 628 may have any of the features of body 128. As shown in FIG. 14, axle 630 may be comprised of a first portion 633a and a second portion 633b. First portion may be shaped like a cone (or frustum). Second portion 633b may be shaped like a portion of a sphere (e.g. a hemisphere). First portion 633a and second portion 633b may be connected that their respective bases, such that axle 630 has a shape similar to an ice cream cone. Bases of first portion 633a and second portion 633b may have the same or similar diameters. Axle 630 may include a cone tip 631 positioned at a narrow end of first portion 633a (at a side of first portion 633a opposite second portion 633b).

Second portion 633b may include a channel 634. Alternatively, channel 634 may be defined through first portion 633a. Channel 634 may include a cylindrical portion 636 and an open side portion 638. An axis B may extend along a central longitudinal axis of cylindrical portion 636. An axis A may be a central longitudinal axis of axle 630 and may extend through a center of first portion 633a and second portion 633b, extending through cone tip 631.

Distal tip 618 may also include a rotatable shaft 674 therein. As most clearly shown in FIG. 15, rotatable shaft 674 may have a shaft portion 675 and may include a projection 676 that extends from one end of shaft portion 675. Projection 676 may extend from a first surface 680 of shaft portion 675. Projection 676 may include a stalk portion 684 and a cylinder portion 686. Stalk portion 684 may connected to first surface 680 and connected to cylinder portion 686 and may extend between first surface 680 and cylinder portion 686. As shown in FIG. 15, first surface 680 may be curved. For example, portions of first surface 680 may have an approximately hemispherical shape. Shaft portion 675 may have a central longitudinal axis X. Axis X may be approximately perpendicular to a longitudinal axis of distal tip 618/device 10 when rotatable shaft 674 is positioned in distal tip 618. Cylinder portion 686 may have a central longitudinal axis Y. Axis Y may be approximately perpendicular to axis X. Rotatable shaft 674 may be rotatable about axis X within distal tip 618. As discussed in further detail below, a housing 621 of distal tip 618 may frictionally interfere with shaft portion 675 as it rotates about axis X. Rotatable shaft 674 may be inhibited from moving in directions other than rotating about axis X. Housing 621 may have any of the features of housing 121.

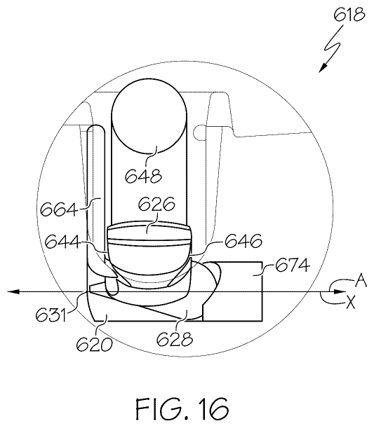

As depicted in FIGS. 16-21B, rotatable shaft 674 may be coupled to axle 630 by inserting projection 676 into channel 634. Axle 630 may be movable relative to rotatable shaft 674. For example, projection 676 may be able to rotate about axis B within channel 634. When rotatable shaft 674 is coupled to axle 630, axis B may be always parallel with axis Y, however axis X may be only parallel with axis A when elevator 626 is not rotated to the left or right about axis B. Cable 664 may be connected to a lateral side 644 or 646 of body 628. For example, as shown in the Figures, cable 664 may be coupled to lateral side 644. Cable 664 may be an only cable coupled to elevator 626. Lateral side 644 may be a side of elevator 626 having cone tip 631 and may be opposite to a side of elevator 626 having second portion 633b. Cable 664 may be connected to and actuated by an elevator lever (e.g. one of levers 60, 62), or otherwise appropriate actuator, of a handle (e.g, handle 12) of the medical device. As discussed above, handle 12 may include only a single lever (referred to herein as lever 60) and the other lever 62 may be omitted.

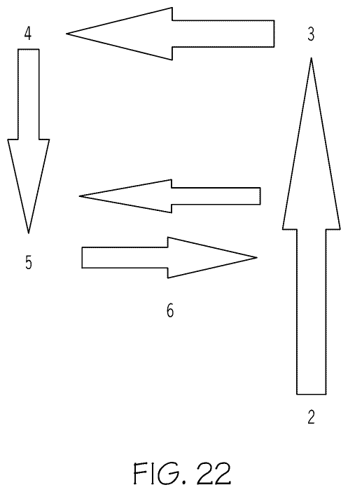

FIGS. 16-21B show cable 664 lifting elevator 626, and FIG. 22 diagrams a path of motion that elevator 626 transitions through in FIGS. 17A-21B as the elevator 626 rotates about axis A and axis B.

FIG. 16 depicts elevator 626 in a lowered (open) configuration. A distal end of cable 664 may be in a maximum distal position relative to distal tip 618 in the lowered configuration. Axis X and axis A may be parallel or coaxial with one another in the lowered configuration. While in the lowered configuration, the elevator may be capable of receiving accessory device 648, and accessory device 648 may extend in a generally distal direction, beyond a distal end of distal tip 618.

FIGS. 17A and 17B depicts elevator 626 being lifted by cable 664. As cable 664 is moved proximally relative to distal tip 618, cable 664 exerts a force on lateral side 644 of elevator. As noted above, the housing 621 of distal tip 618 may frictionally interfere with/frictionally engage with shaft portion 675. Projection 676 may freely move relative to channel 634 without such frictional interference. Thus, as cable 664 moves proximally, elevator 626 may first pivot about axis B. As elevator 626 pivots generally proximally about axis B, elevator 626 rotates to the right when viewed in the proximal direction (i.e., medially, toward an imaging device 13 of device 10), thereby deflecting accessory device 648 to the right as well. As shown in FIG. 17A, in particular, axis A may be offset from axis X as elevator 626 pivots about axis B. Cone tip 631 may move within a cavity 620 (having any of the properties of cavities 420, 320, 120 of housing 621. The cavity 620 of housing 621 may limit an amount that elevator 626 may pivot about axis B, until elevator 626 has reached a fully right-pivoted position. In the configuration of FIGS. 17A and 17B, accessory device 648 may be outside a field of view of an imaging device 13 of device 10.

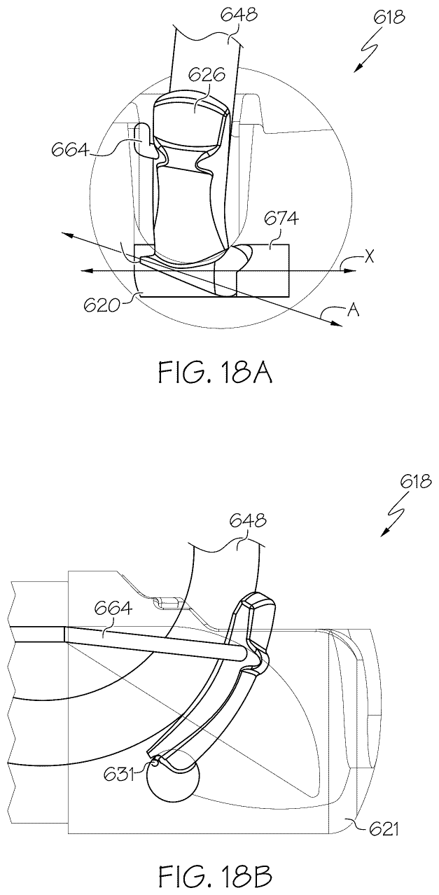

As cable 664 continues move proximally, after elevator 626 has reached a fully right-pivoted position, a force of cable 664 may then overcome the frictional force between housing 621 and shaft portion 675, rotating shaft portion 675. Thus, elevator 626 may be rotated in the proximal direction about axis X. Accessory device 648 may enter a field of view of an imaging device 13 of device 10 as elevator 626 rotates upward about axis A. In general, rotating elevator 626 about axis X may be used to raise and lower elevator 626, causing accessory device 648 to be deflected in an up/down direction of the Figures and of a field of view of an imaging device 13 of medical device 10. Elevator 626 may reach a maximum pitch (e.g., when elevator 626 interacts with a portion of housing 621 such as a boss having any property of boss 124, discussed above). FIGS. 18A and 18B illustrate such a raised/closed position of elevator 626. As shown in FIGS. 18A and 18B, axis A may remain offset from axis X. The motion of elevator 626 from the configuration of FIGS. 17A and 17B to the configuration of FIGS. 18A and 18B is illustrated by the arrow from position 2 (FIGS. 17A and 17B) to position 3 (FIGS. 18A and 18B) in FIG. 22.

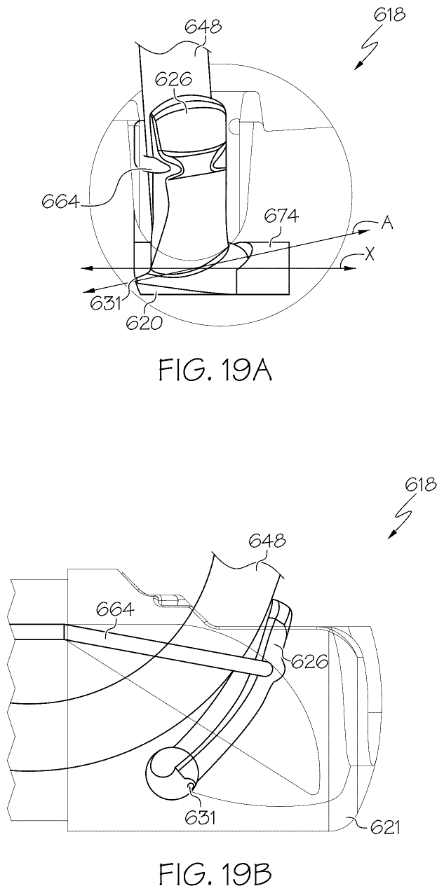

If cable 664 is loosened (e.g., moved distally), then, as shown in FIGS. 19A and 19B (elevator 626 may be deflected to the left (away from an imaging device 13 of device 10). Because of the friction between housing 621 and shaft portion 675 and the lack of friction between projection 676 and channel 634, elevator 626 may pivot about axis B without rotating about axis X. In other words, projection 676 may rotate/pivot within channel 634 without shaft portion 675 rotating with respect to housing 621. Thus, elevator 626 may deflect accessory device 648 from right to left. This motion is illustrated as the arrow from position 3 (FIGS. 18A and 18B) to position 4 (FIGS. 19A and 19B) in FIG. 22. Cone tip 631 may move within the cavity 620 of housing 621. As discussed above, the cavity 620 of housing 621 may limit an amount that elevator 626 may pivot about axis B, until elevator 626 has reached a fully left-pivoted position (FIGS. 19A and 19B). As shown in FIGS. 19A and 19B, axis A may be offset from axis X in an opposite direction to FIGS. 17A-18B, above. It will be appreciated that, as elevator 626 transitions from the configuration of FIGS. 18A and 18B to the configurations of FIGS. 19A and 19B, elevator 626 may pass through (and optionally retained) in a neutral configuration in which axis X is parallel to or coaxial with axis A.

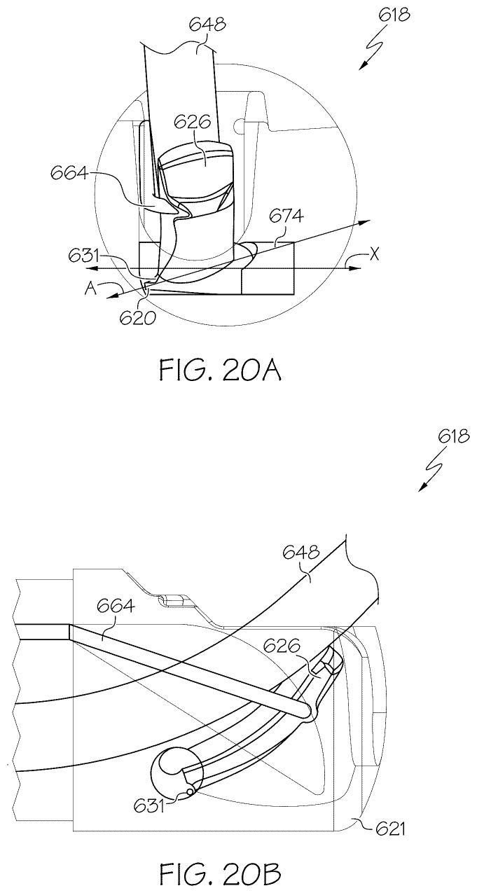

As cable 664 continues to move distally, after elevator 626 has reached a fully left-pivoted position, a force of cable 664 may then overcome the frictional force between housing 621 and shaft portion 675, rotating shaft portion 675. Thus, as shown in FIGS. 20A and 20B, elevator 626 may be rotated in the distal direction about axis X to lower or open elevator 626. As cable 664 continues to move distally, elevator 626 remains pivoted leftward (away from an imaging device 13 of device 10). Thus, as shown in FIGS. 20A and 20B, axis X may remain offset from axis A. The lowering of elevator 326 is illustrated as the arrow from position 4 (FIGS. 19A and 19B) to position 5 (FIGS. 20A and 20B) in FIG. 22.

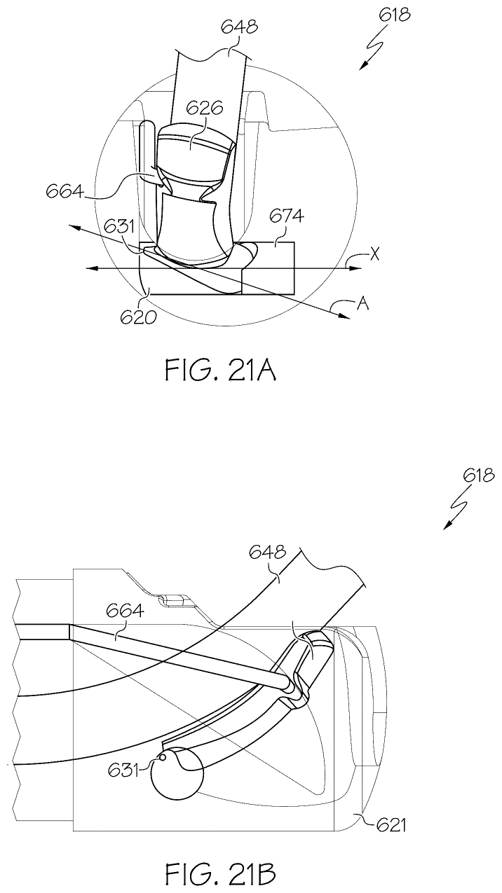

As shown in FIGS. 21A and 21B, if cable 664 is proximally moved before elevator 626 has returned to a fully the lowered configuration, then elevator 626 will pivot about axis B (because of the lack of friction between projection 676 and channel 634), deflecting accessory device 648 from left to right. If cable 664 is moved distally, then elevator 626 may again pivot about axis B, deflecting accessory device from right to left. After elevator 626 pivots about axis B to a fully right-pivoted position, elevator 626 may again begin ascending. During ascension or descension, an operator may move lever 60 to move cable 664 proximally or distally, thereby deflecting elevator 626 and accessory device 648 right or left, as discussed above. The arrows labeled 6 in FIG. 22 depict this rightward/leftward movement along a raising/lowering path of elevator 626.

It should be understood that the movement of elevator 626 may be somewhat cyclical and may move through the configurations of FIGS. 17A through 21B in order until the elevator position is locked or the elevator is allowed to descend back to the lowered configuration.

While principles of this disclosure are described herein with reference to illustrative examples for particular applications, it should be understood that the disclosure is not limited thereto. Those having ordinary skill in the art and access to the teachings provided herein will recognize additional modifications, applications, embodiments, and substitution of equivalents all fall within the scope of the features described herein. Accordingly, the claimed features are not to be considered as limited by the foregoing description.

What is claimed is:

1. A medical device comprising:
a handle; and
a distal tip, the distal tip including an elevator configured to change an orientation of an accessory device, wherein the elevator is rotatable about a first axis and a second axis, wherein the first axis differs from the second axis.

2. The medical device of claim 1, wherein rotation of the elevator about the first axis raises and lowers the elevator.

3. The medical device of claim 1, wherein rotation of the elevator about the second axis moves the elevator about a longitudinal axis of a body of the elevator.

4. The medical device of claim 1, further comprising a first wire or cable coupled to a first side of the elevator and a second wire or cable coupled to a second side of the elevator.

5. The medical device of claim 4, wherein movement of the first wire or cable relative to the second wire or cable or rotates the elevator about the second axis.

6. The medical device of claim 4, further comprising a roll bar, a third wire coupled to the roll bar, and a fourth wire coupled to the roll bar, wherein movement of the third wire is configured to rotate the elevator about the first axis, and wherein movement of the fourth wire is configured to rotate the elevator about the second axis.

7. The medical device of claim 6, wherein the first wire is coupled to the roll bar at a first connection point, wherein the second wire is coupled to the roll bar at a second connection point, wherein the third wire is coupled to the roll bar at a third connection point, and wherein the third connection point is equidistant from the first connection point and the second connection point.

8. The medical device of claim 4, wherein the handle includes a first actuator coupled to the first wire or cable and a second actuator coupled to the second wire or cable, wherein the first actuator and the second actuator are actuatable independently of one another.

9. The medical device of claim 1, wherein proximal or distal movement of a single wire is configured to rotate the elevator about both the first axis and the second axis.

10. The medical device of claim 8, wherein the elevator includes an axle defining a channel, wherein the distal tip further includes a rotatable shaft having a projection, and wherein the channel is configured to receive the projection.

11. The medical device of claim 10, wherein the first axis is a longitudinal axis of a shaft of the rotatable shaft, and wherein the second axis is a longitudinal axis of (a) the projection or (b) the channel.

12. The medical device of claim 1, wherein the elevator includes an approximately biconical axle.

13. The medical device of claim 9, wherein the distal tip includes a housing defining a cavity, wherein the elevator includes an axle received within the cavity, and wherein the cavity has an approximately cylindrical shape.

14. The medical device of claim 1, wherein the elevator includes an axle, wherein the distal tip includes a housing defining a cavity, wherein the axle is received within the cavity, wherein the cavity is configured to allow a central portion of the axle to rotate about a longitudinal axis of the axle and to prevent the central portion of the axle from translating within the cavity.

15. The medical device of claim 14, wherein the cavity is configured to allow ends of the axle to translate within the cavity.

16. A medical device comprising:
a distal tip, the distal tip including an elevator configured to change an orientation of an accessory device; and
a handle, the handle including a first actuator coupled to a first control wire or cable and a second actuator coupled to a second control wire or cable, wherein each of the first actuator and the second actuator is configured to rotate the elevator about at least one axis.

17. The medical device of claim 16, wherein the first control wire or cable is coupled to a first side of the elevator, and wherein the second control wire or cable is coupled to a second side of the elevator.

18. The medical device of claim 16, wherein each of the first actuator and the second actuator is separately actuatable.

19. A distal tip of a medical device comprising:
an elevator having an axle and a body; and
at least one wire or cable coupled to the elevator;
wherein the at least one wire or cable is configured to rotate the elevator about a longitudinal axis of the axle and about a longitudinal axis of the body.

20. The distal tip of claim 19, wherein the at least one wire or cable is at least two wires or cables.
